(19) United States
(12) Patent Application Publication
(10) Pub. No.: US 2026/0063415 A1
(43) Pub. Date: Mar. 5, 2026

(54) DEPTH SENSING SYSTEM AND METHOD THEREOF

(71) Applicant: HIMAX TECHNOLOGIES LIMITED, Tainan City (TW)

(72) Inventor: Chin-Jung Tsai, Tainan City (TW)

(73) Assignee: HIMAX TECHNOLOGIES LIMITED, Tainan City (TW)

(21) Appl. No.: 18/822,279

(22) Filed: Sep. 2, 2024

(51) Int. Cl. G01B 11/22; G06T 7/521; G06T 7/77

(52) U.S. Cl. CPC G01B 11/22; G06T 7/521; G06T 7/77; G06T 2207/10028

(57) ABSTRACT

A depth sensing system includes a light-emitting device, a sensing module and a controller. The light-emitting device is configured to emit a light beam toward to a scene. The sensing module is configured to receive a reflective beam reflected from the scene to generate a scene image. The controller is electrically connected to the light-emitting device and the sensing module. The controller is configured to calculate an IQ image of the scene according to the scene image. The controller is configured to calculate confidence values of each pixel of the IQ image to generate a confidence image. The controller is configured to calculate a calibrated IQ image according to the confidence values, and then calculate a depth image of the scene. A depth sensing method is also provided.

### BACKGROUND

### Technical Field

The invention generally relates to a sensing system and method thereof and, in particular, to a depth sensing system and method thereof.

### Description of Related Art

The present depth sensing systems includes time of flight (ToF) and indirect TOF (iToF) in terms of technology. The ToF measures the depth of the scene by directly measuring the time differences of a light beam traveling from the depth sensing system to the scene and then reflecting back from the scene to the depth sensing system. On the contrary, iToF indirectly measures the depth of the scene by measuring the difference (such as the phase difference) of the light beam emitted from the depth sensing system and the reflective beam reflected from the scene back to the depth sensing system.

However, although iToF has the advantages of high reliability of depth reproduction and high resolution, it still has the following problem. When there is a near-field high reflective object in the scene, the reflective beam will be reflected multiple times between the lens module and the sensor due to its higher light intensity, resulting in inaccurate depth measurement of the surrounding objects. That is, the lens flare problem. Moreover, although the flare point spread function (PSF) generated by this object can be measured and the flare effect can be eliminated by deconvolution, in practice, this flare PSF has the characteristics of a long tail (large kernel), and small intensity, so it is difficult to be well estimated and perform deconvolution or requires expensive costs to perform deconvolution. Furthermore, the aforementioned problem is more serious when the background in the scene has low reflectivity.

### SUMMARY

Accordingly, the invention is directed to a depth sensing system and method thereof, which could provide the effective process of flare cancellation and further reduce system costs.

According to an embodiment of the disclosure, a depth sensing system includes a light-emitting device, a sensing module and a controller. The light-emitting device is configured to emit a light beam toward to a scene. The sensing module is configured to receive a reflective beam reflected from the scene to generate a scene image. The controller is electrically connected to the light-emitting device and the sensing module. The controller is configured to calculate an IQ image of the scene according to the scene image. The controller is configured to calculate confidence values of each pixel of the IQ image to generate a confidence image. The controller is configured to calculate a calibrated IQ image according to the confidence values, and then calculate a depth image of the scene.

According to an embodiment of the disclosure, a depth sensing method includes the following steps. Emitting a light beam toward to a scene. Receiving a reflective beam reflected from the scene to generate a scene image. Calculating an IQ image of the scene according to the scene image. Calculating confidence values of each pixel of the IQ image to generate a confidence image. Calculating a calibrated IQ image according to the confidence values, and then calculating a depth image of the scene.

Based on the above, according to an embodiment of the disclosure, in the depth sensing system and depth sensing method, the confidence values of each pixel of the IQ image are calculated to generate the confidence image, the calibrated IQ image is calculated according to the confidence values, and then the depth image of the scene is calculated. Thus, the process of the calibration by calculating the confidence values could play a similar role as the process of deconvolution by considering the flare source as PSF, and the process of the calibration in the disclosure will be more effective, and therefore further reduce system costs.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a block view of a depth sensing system according to an embodiment of the disclosure.

FIG. 2A is a schematic diagram of the depth sensing system for sensing toward a scene according to an embodiment of the disclosure.

FIG. 2B is a schematic diagram of a phase image of the scene obtained by the depth sensing system in FIG. 2A.

FIG. 2C is a schematic diagram of a calibrated phase image after calibration of the confidence image in FIG. 2B.

FIG. 2D is a schematic diagram of a confidence image of the scene obtain by the depth sensing system in FIG. 2A.

FIG. 3 is a schematic diagram of block BL1 in the confidence image of FIG. 2D by calculating its confidence histogram.

FIG. 4 is a flow chart of a depth sensing method according to an embodiment of the disclosure.

FIG. 5 is a detailed flow chart of FIG. 4.

FIG. 6 is a detailed flow chart of a depth sensing method according to another embodiment of the disclosure.

### DESCRIPTION OF THE EMBODIMENTS

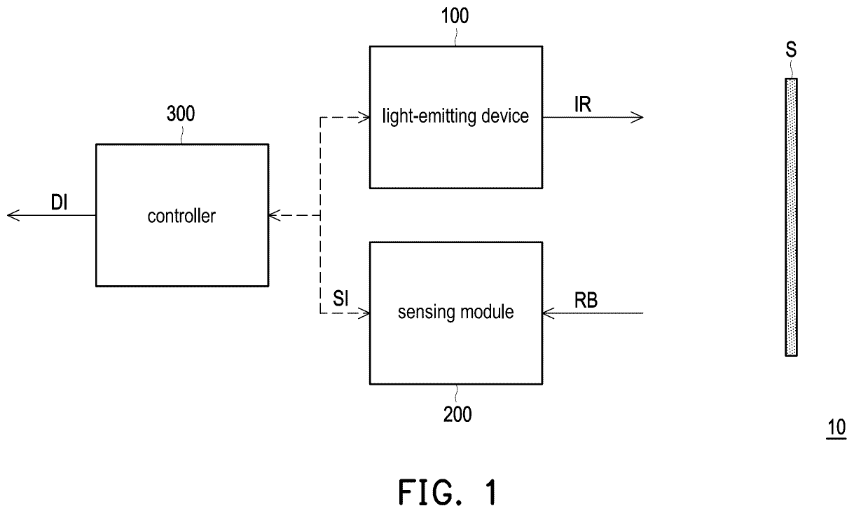

FIG. 1 is a block view of a depth sensing system according to an embodiment of the disclosure Referring to FIG. 1, an embodiment of the disclosure provides a depth sensing system 10, which includes a light-emitting device 100, a sensing module 200 and a controller 300.

In this embodiment, the light-emitting device 100 is configured to emit a light beam IB toward to a scene S. The light-emitting device 100 may be light-emitting diodes (LEDs) or laser diodes (LDs). The light beam IB could be the IR light beam, but the disclosure is not limited thereto.

In this embodiment, the sensing module 200 is configured to receive a reflective beam RB (of the light beam IB) reflected from the scene S to generate a scene image SI. The sensing module 200 may include a sensor and a lens module. The sensor may be optical sensors, such as complementary metal-oxide semiconductors (CMOS), but the disclosure is not limited thereto. The reflective beam RB from the scene S passes through the lens module 200, and is incident on the sensor.

In this embodiment, the controller 300 includes, for example, a central processing unit (CPU), a microprocessor, a digital signal processor (DSP), a programmable controller, a programmable logic device (PLD), or other similar devices, or a combination of the said devices, which are not particularly limited by the disclosure. Further, in an embodiment, each of the functions performed by the controller 300 may be implemented as a plurality of program codes. These program codes will be stored in a memory, so that these program codes may be executed by the controller 300. Alternatively, in an embodiment, each of the functions performed by the controller 300 may be implemented as one or more circuits. The disclosure is not intended to limit whether each of the functions performed by the controller 300 is implemented by ways of software or hardware.

In this embodiment, the controller 300 is electrically connected to the light-emitting device 100 and the sensing module 200. The controller 300 is configured to calculate an IQ image of the scene S according to the scene image SI. The controller 300 is configured to calculate confidence values of each pixel of the IQ image to generate a confidence image. The controller 300 is configured to calculate a calibrated IQ image according to the confidence values, and then calculate a depth image DI of the scene S.

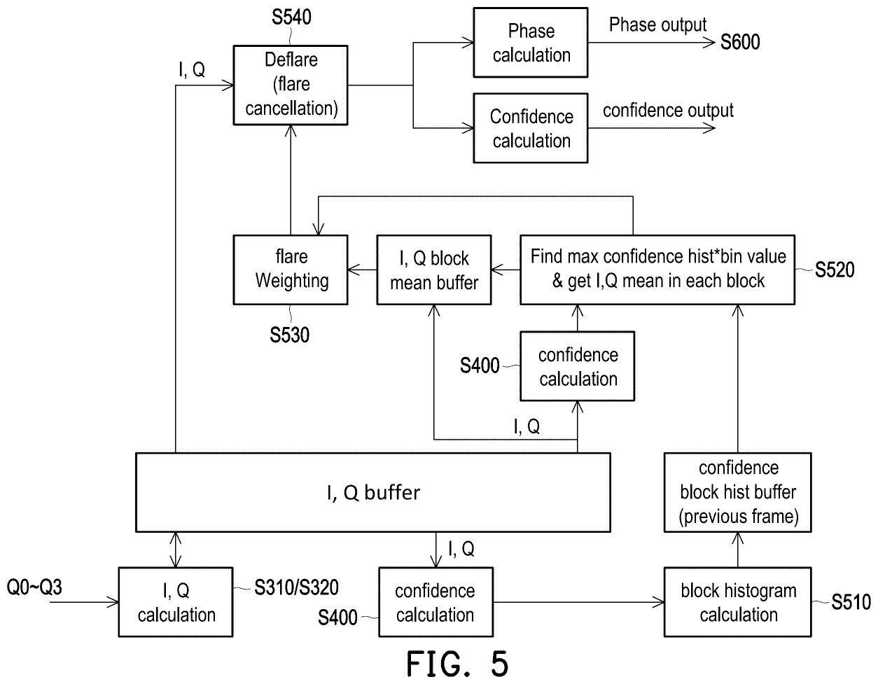

Specifically, in this embodiment, the controller 300 calculates a waveform of each pixel of the scene image SI by comparing phase differences of 0, 90, 180 and 270 degrees (data Q0 to Q3 in FIG. 5) between the light beam IB and the reflective beam RB (step 310 in FIG. 5). The controller 300 projects the waveforms to a complex plane to obtain an I-value and a Q-value of each pixel of the IQ image, wherein the I-value and the Q-value are respectively a real part and an imaginary part of the waveform in the complex plane (step 320 in FIG. 5). That is, each pixel of the IQ image is a 2D value (I-value, Q-value), and the angle of the vector formed by the I-value and the Q-value in the complex plane represents the phase of the pixel/waveform.

For example, the phase difference of a pixel of the scene S could be calculated by the following equation:

$$Δ φ = {tan}^{- 1} ( \frac{{Q}_{1} - {Q}_{3}}{{Q}_{0} - {Q}_{2}} )$$

where Δφ is the phase difference, Q0 is the signal of 0 degree of reflective beam RB, Q1 is the signal of 90 degree of reflective beam RB, Q2 is the signal of 180 degree of reflective beam RB, and Q3 is the signal of 270 degree of reflective beam RB. Thus, the phase difference of the scene S forms the phase image, and the depth of the pixel could be calculated by the following equation:

$$D = \frac{c}{2} ( \frac{Δ φ}{2 π f} )$$

where D is the depth, c is the velocity of light, and f is the frequency of the light beam IB.

Moreover, the aforementioned confidence value is \sqrt{I²+Q²}, where I is the I-value and Q is the Q-value. That is, the controller 300 transforms the IQ image into the phase image and the confidence image, and stores the confidence image in the buffer. Moreover, the value of the confidence value could represent the strength of the signal of the pixel. Thus, the larger value of the confidence value, the higher strength of signal of the pixel.

In this embodiment, the controller 300 divides the confidence image into a plurality of blocks, counts a distribution of the confidence values in each block to obtain a confidence histogram of each block, and finds out a flare source in each block (step 510 to step 520 in FIG. 5). Moreover, the flare source is a mean of bins in each histogram that has the maximum value of confidence value multiplied with its numbers. This block histogram could shrink down PSF kernel size (spatial domain) and dynamic range (amplitude domain). Further, the ones would be selected for deflare processing when the confidence ratios of the flare sources to that of the current pixel are larger than the threshold. For example, the confidence image could be an image of 640 pixel×480 pixel. Each block could be set as 16 pixel×16 pixel. Thus, the confidence image is divided into 40*30 blocks.

In this embodiment, the controller 300 determines weightings of the flare sources according to a weighting table (step 530 in FIG. 5). In practice, the weighting LUT w_{t,i} could be according to both the histogram bin, numbers and spatial coordinate. The weighting table could be stored in the memory of the controller 300, but the disclosure is not limited thereto. The controller 300 obtains calibrated pixels of the calibrated IQ image according to the weightings and the flare sources (step 540 in FIG. 5), where the calibrated pixel satisfy:

$${I}^{′} + {jQ}^{′} = ( I + Q ) - {\sum }_{i} ( {I}_{F , i} + j {Q}_{F , i} ) \times {w}_{t , i}$$

wherein I′ and Q′ are respectively a I-value and a Q-value of the calibrated pixel, I and Q are respectively a I-value and a Q-value of a corresponding pixel in the block corresponding to the calibrated pixel, I_{F,i} and Q_{F,i} are respectively I-values and Q-values of the flare sources in the block and its neighboring blocks corresponding to the calibrated pixel, and w_{t,i} are the weightings of the flare sources in the block and its neighboring blocks corresponding to the calibrated pixel.

The aforementioned neighboring blocks of a block, for example, could be defined as 10 nearby blocks (but the disclosure is not limited thereto) of this block. Thus, in this embodiment, the process of aforementioned cancellation by considering the weightings of this block and its neighboring blocks could play a similar role as the process of deconvolution by considering the flare source as PSF. Furthermore, the process of the cancellation in the disclosure will be more effective, and therefore further reduce system costs.

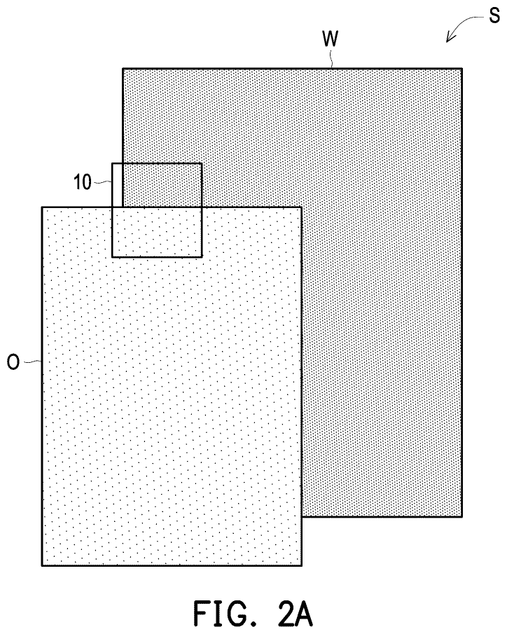

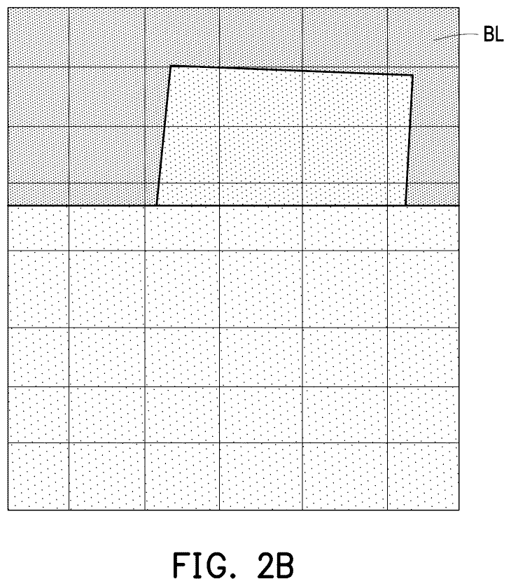

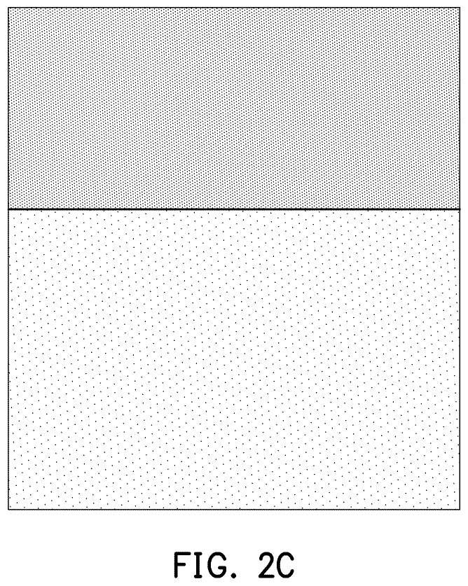

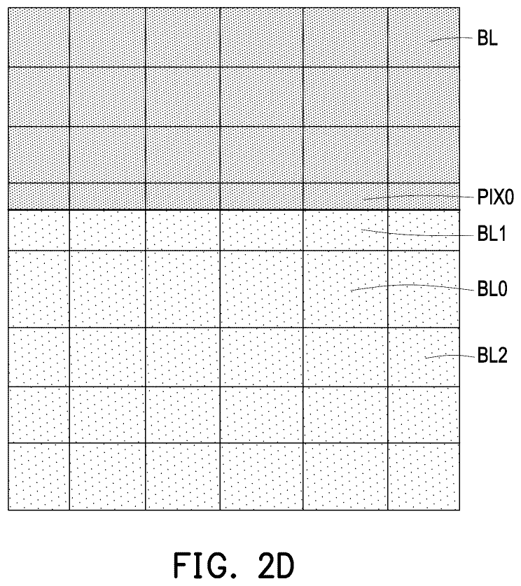

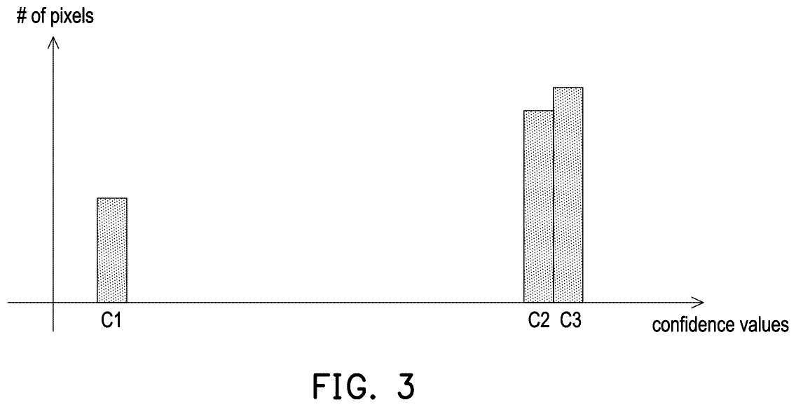

FIG. 2A is a schematic diagram of the depth sensing system for sensing toward a scene according to an embodiment of the disclosure. FIG. 2B is a schematic diagram of a phase image of the scene obtained by the depth sensing system in FIG. 2A. FIG. 2C is a schematic diagram of a calibrated phase image after calibration of the confidence image in FIG. 2B. FIG. 2D is a schematic diagram of a confidence image of the scene obtain by the depth sensing system in FIG. 2A. FIG. 3 is a schematic diagram of block BL1 in the confidence image of FIG. 2D by calculating its confidence histogram.

In FIG. 2A, the depth sensing system 10 senses toward the scene S. The scene S includes objects O and W, but the disclosure does not limit the number of objects in the scene S. The object O could be a white board with high reflectivity. The object W could be a background wall with low reflectivity. In FIG. 2B, the upper part could represent the signal from the object W, and the lower part could represent the signal from the object O. Moreover, the confidence image of FIG. 2D is further divided into multiple blocks BL. It is obviously that for PIX0, the signals of several blocks could be considered as the flare signal, such as blocks BL0, BL1, and BL2.

In FIG. 3, the confidence histogram of block BL1 shows the signals of block BL1 are contributed by three bins C1, C2 and C3. The signal of bin C1 could be contributed from the object W, but the signals of bins C2 and C3 may be the flare source contributed from the object O. Thus, the controller 300 could take the mean of bins C3 (which is with the maximum value of confidence value multiplied with numbers) as the flare source of block BL1 to calibrate the signal of bin C1. Finally, by choosing proper weightings of block BL1 and its neighboring blocks BL, the calibrated IQ image could be obtained as shown in FIG. 2C.

In another embodiment, the controller 300 marks a pixel in each block BL as an unknown pixel if a difference between a confidence value of the flare source and the confidence value of the pixel is larger than a threshold. For example, in FIG. 3, the signal of bin C1 is much weaker than the signals of bins C2 and C3. Thus, the calibration of pixels corresponding to bin C1 might still be less accurate. The controller 300 could mark the pixels corresponding to bin C1 as the unknown pixels.

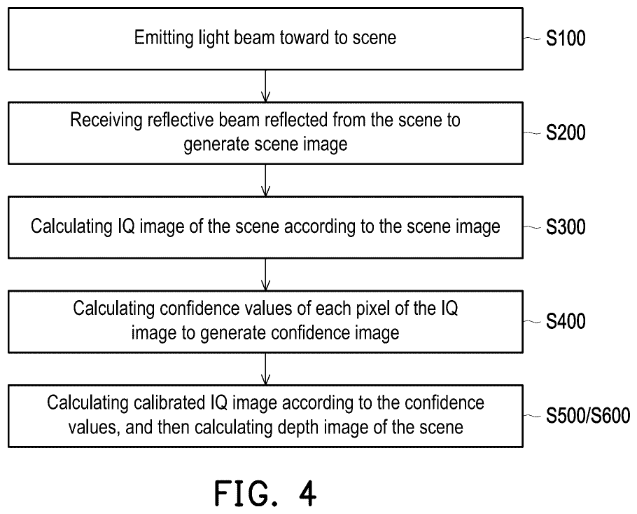

FIG. 4 is a flow chart of a depth sensing method according to an embodiment of the disclosure. Referring to FIG. 4, an embodiment of the disclosure provides a depth sensing method includes the following steps. In step S100, emitting a light beam IB toward to a scene S. In step S200, receiving a reflective beam RB reflected from the scene S to generate a scene image SI. In step S300, calculating a IQ image of the scene S according to the scene image Si. In step S400, calculating confidence values of each pixel of the IQ image to generate a confidence image. In steps S500 and S600, calculating a calibrated IQ image according to the confidence values, and then calculating a depth image DI of the scene S.

FIG. 5 is a detailed flow chart of FIG. 4. Referring to FIG. 5, in this embodiment, the above-mentioned Step S500 includes the following steps. In step S310, calculating a waveform of each pixel of the scene image SI by comparing phase differences of 0, 90, 180 and 270 degrees between the light beam IB and the reflective beam RB. In step S320, projecting the waveforms to a complex plane to obtain an I-value and a Q-value of each pixel in the IQ image.

In this embodiment, the above-mentioned Step S500 includes the following steps. In steps S510 and S520, dividing the confidence image into a plurality of blocks BL, counting a distribution of the confidence values in each block BL to obtain a confidence histogram of each block BL, and finding out a flare source in each block BL. In step S530, determining weightings of the flare sources according to a weighting table. In step S540, obtaining calibrated pixels of the calibrated IQ image according to the weightings and the flare sources.

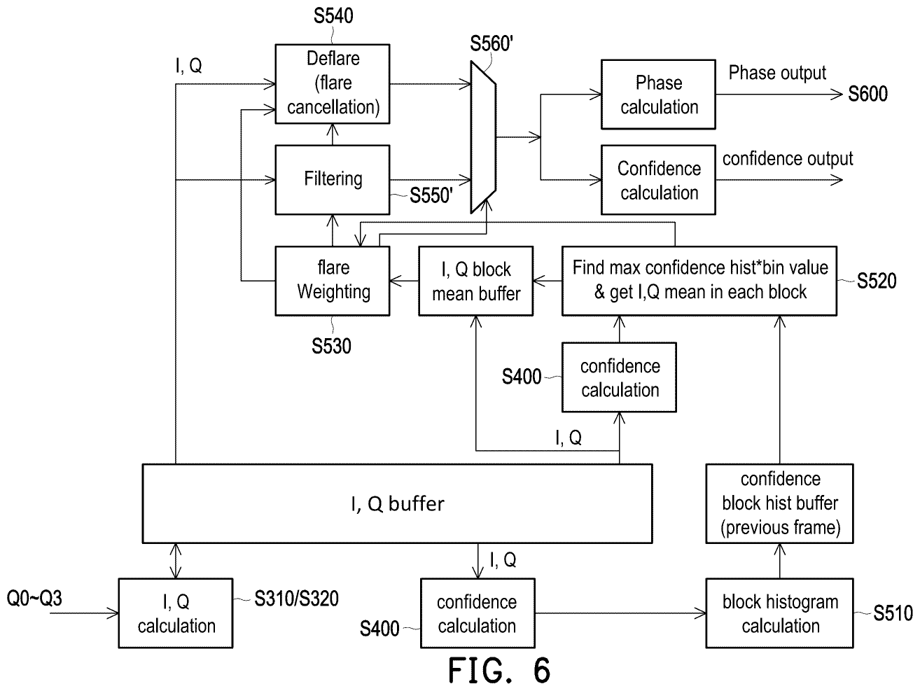

FIG. 6 is a detailed flow chart of a depth sensing method according to another embodiment of the disclosure. Referring to FIG. 6, in another embodiment, the above-mentioned Step S500 further includes the following step. In step S550′, marking a pixel in each block BL as an unknown pixel if a difference between a confidence value of the flare source and the confidence value of the pixel is larger than a threshold. In step S560′, determining to go through step 540 or step 550′ (by using the multiplexer/controller 300).

To sum up, according to an embodiment of the disclosure, in the depth sensing system and depth sensing method, the IQ image of the scene is calculated according to the scene image.

The confidence values of each pixel of the IQ image are calculated to generate the confidence image. The calibrated IQ image is calculated according to the confidence values, and then the depth image of the scene is calculated. Thus, the process of the calibration by calculating the confidence values could play a similar role as the process of deconvolution by considering the flare source as PSF, and the process of the calibration in the disclosure will be more effective, and therefore further reduce system costs.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A depth sensing system, comprising:
a light-emitting device, configured to emit a light beam toward to a scene;
a sensing module, configured to receive a reflective beam reflected from the scene to generate a scene image; and
a controller, electrically connected to the light-emitting device and the sensing module,
wherein the controller is configured to calculate a IQ image of the scene according to the scene image;
wherein the controller is configured to calculate confidence values of each pixel of the IQ image to generate a confidence image; and
wherein the controller is configured to calculate a calibrated IQ image according to the confidence values, and then calculate a depth image of the scene.

2. The depth sensing system according to claim 1,
wherein the controller calculates a waveform of each pixel of the scene image by comparing phase differences of 0, 90, 180 and 270 degrees between the light beam and the reflective beam; and
wherein the controller projects the waveforms to a complex plane to obtain an I-value and a Q-value of each pixel in the IQ image, wherein the I-value and the Q-value are respectively a real part and an imaginary part of the waveform in the complex plane,
wherein the confidence value is \sqrt{I²+Q²}, where I is the I-value and Q is the Q-value.

3. The depth sensing system according to claim 1,
wherein the controller divides the confidence image into a plurality of blocks, counts a distribution of the confidence values in each block to obtain a confidence histogram of each block, and finds out a flare source in each block.

4. The depth sensing system according to claim 3, wherein the flare source is a mean of bins in each histogram that has a maximum value of a confidence value multiplied with its numbers.

5. The depth sensing system according to claim 3,
wherein the controller determines weightings of the flare sources according to a weighting table; and
wherein the controller obtains calibrated pixels of the calibrated IQ image according to the weightings and the flare sources, where the calibrated pixel satisfy:
$${I}^{′} + {jQ}^{′} = ( I + Q ) - {\sum }_{i} ( {I}_{F , i} + j {Q}_{F , i} ) \times {w}_{t , i}$$
wherein I′ and Q′ are respectively a I-value and a Q-value of the calibrated pixel, I and Q are respectively a I-value and a Q-value of a corresponding pixel in the block corresponding to the calibrated pixel, I_{F,i} and Q_{F,i} are respectively I-values and Q-values of the flare sources in the block and its neighboring blocks corresponding to the calibrated pixel, and w_{t,i} are the weightings of the flare sources in the block and its neighboring blocks corresponding to the calibrated pixel.

6. The depth sensing system according to claim 3,
wherein the controller marks a pixel in each block as an unknown pixel if a difference between a confidence value of the flare source and the confidence value of the pixel is larger than a threshold.

7. A depth sensing method, comprising:
emitting a light beam toward to a scene;
receiving a reflective beam reflected from the scene to generate a scene image;
calculating an IQ image of the scene according to the scene image;
calculating confidence values of each pixel of the IQ image to generate a confidence image; and
calculating a calibrated IQ image according to the confidence values, and then calculating a depth image of the scene.

8. The depth sensing method according to claim 7, wherein the step of calculating the IQ image of the scene according to the scene image comprises:
calculating a waveform of each pixel of the scene image by comparing phase differences of 0, 90, 180 and 270 degrees between the light beam and the reflective beam; and
projecting the waveforms to a complex plane to obtain an I-value and a Q-value of each pixel in the IQ image, wherein the I-value and the Q-value are respectively a real part and an imaginary part of the waveform in the complex plane
wherein the confidence value is \sqrt{I²+Q²}, where I is the I-value and Q is the Q-value.

9. The depth sensing method according to claim 7, wherein the step of calculating the calibrated IQ image according to the confidence values, and then calculating the depth image of the scene comprises:
dividing the confidence image into a plurality of blocks, counting a distribution of the confidence values in each block to obtain a confidence histogram of each block, and finding out a flare source in each block.

10. The depth sensing method according to claim 9, wherein the flare source is a mean of bins in each histogram that has a maximum value of a confidence value multiplied with its numbers.

11. The depth sensing method according to claim 9, wherein the step of calculating the calibrated IQ image according to the confidence values, and then calculating the depth image of the scene further comprises:
determining weightings of the flare sources according to a weighting table; and
obtaining calibrated pixels of the calibrated IQ image according to the weightings and the flare sources, where the calibrated pixel satisfy:
$${I}^{′} + {jQ}^{′} = ( I + Q ) - {\sum }_{i} ( {I}_{F , i} + j {Q}_{F , i} ) \times {w}_{t , i}$$
wherein I′ and Q′ are respectively a I-value and a Q-value of the calibrated pixel, I and Q are respectively a I-value and a Q-value of a corresponding pixel in the block corresponding to the calibrated pixel, I_{F,i} and Q_{F,i} are respectively I-values and Q-values of the flare sources in the block and its neighboring blocks corresponding to the calibrated pixel, and w_{t,i} are the weightings of the flare sources in the block and its neighboring blocks corresponding to the calibrated pixel.

12. The depth sensing method according to claim 9, wherein the step of calculating the calibrated IQ image according to the confidence values, and then calculating the depth image of the scene further comprises:
marking a pixel in each block as an unknown pixel if a difference between a confidence value of the flare source and the confidence value of the pixel is larger than a threshold.
